(19) United States
(12) Patent Application Publication
(10) Pub. No.: US 2013/0060204 A1
(43) Pub. Date: Mar. 7, 2013

(54) Syringe with prevention of backflow of blood

(71) Applicant: Kuo-Cheng WU, Kaohsiung City (TW)

(72) Inventor: Kuo-Cheng WU, Kaohsiung City (TW)

(21) Appl. No.: 13/224,387

(22) Filed: Sep. 2, 2011

(51) Int. Cl. A61M 5/315

(52) U.S. Cl. 604/236

(57) ABSTRACT

A syringe includes a hollow barrel and a liquid pushing assembly. The barrel has an injection end. The liquid pushing assembly includes a plunger and an elastic jacket. The plunger includes a stopper on a front end thereof. The stopper includes an abutment face. The plunger is received in the barrel. The abutment face of the stopper faces the injection end of the barrel. The elastic jacket includes a through-hole extending along an axis. The elastic jacket is mounted around the plunger. The abutment face of the stopper is exposed outside of the through-hole of the elastic jacket.

### BACKGROUND OF THE INVENTION

1. Field of the Invention

The present invention relates to a syringe and, more particularly, to a syringe that receives a medication and that can be used in an intravenous drip while preventing backflow of blood.

2. Description of the Related Art

FIG. 1 shows a conventional syringe 9 including a barrel 91, a plunger 92, and an elastic jacket 93. The barrel 91 is hollow for receiving an injection. The barrel 91 includes an injection end 911 to which a needle cannula 94 is mounted. An opening 912 is defined in the other end of the barrel 91. A thumb rest 921 is provided on an end of the plunger 92 and can be pushed by a user. A stopper 922 is mounted to the other end of the plunger 92. The jacket 93 is elastic and engaged around the stopper 922 to enclose a front end of the stopper 922. An outer diameter of the elastic jacket 93 is slightly larger than an inner diameter of the barrel 91.

The plunger 92 is inserted into the barrel 91. The elastic jacket 93 is slightly compressed inward in a radial direction and abuts with an inner periphery of the barrel 91. The thumb rest 921 of the plunger 92 extends beyond the opening 912 of the barrel 91. The user can push the thumb rest 921 to move the elastic jacket 93 relative to the inner periphery of the barrel 91, pushing the injection to pass through the injection end 911 and the needle cannula 94 into a body of a patient.

Due to elasticity of the elastic jacket 93, when the elastic jacket 93 is pushed to a position adjacent to the injection end 911 of the barrel 91 during an intravenous injection, the front end of the elastic jacket 93 is deformed by the inner periphery of the barrel 91 adjacent to the injection end 911. When the user stops pushing the thumb rest 921, the elasticity of the elastic jacket 93 generates suction force in the needle cannula 94. This suction force creates a negative pressure that draws a small amount of blood from the blood vessel of the patient, causing backflow of blood.

The backflow of blood in an ordinary hypodermic injection would not cause injury. However, if the backflow of blood occurs in an intravenous drip, the backflowing blood is apt to reside in a guiding tube connected to a needle and gradually congeals. The blood clot formed by congelation of the blood may block the guiding tube and may even enter the blood vessel of the patient in a subsequent injection or intravenous drip, leading to the risk of blockage of the blood vessel of the patient.

Furthermore, in assembly of the conventional syringe 9, the front end of the stopper 922 is squeezed into the elastic jacket 93 that later resumes its shape. To allow easy engagement between the elastic jacket 93 and the stopper 922, the elastic jacket 93 generally has an inner space larger than the volume of the stopper 922 to provide loose fitting.

However, many gaps G exist between an inner periphery of the elastic jacket 93 and the stopper 922 due to the loose fitting therebetween. These gaps G will increase the axial deformation of the elastic jacket 93 when squeezed by the inner periphery adjacent to the injection end 911, increasing the risk of backflow of blood.

### SUMMARY OF THE INVENTION

An objective of the present invention is to provide a syringe with prevention of backflow of blood including a stopper having an exposed front end to abut with the inner periphery of the injection end of the barrel, avoiding backflow of blood during an injection.

Another objective of the present invention is to provide a syringe with prevention of backflow of blood including a stopper having a front end abutting with the inner periphery of the injection end of the barrel, avoiding generation of axial gaps between the stopper and the elastic jacket.

The present invention fulfills the above objectives by providing, in an aspect, a syringe including a hollow barrel and a liquid pushing assembly. The barrel has an injection end. The liquid pushing assembly includes a plunger and an elastic jacket. The plunger includes a stopper on a front end thereof. The stopper includes an abutment face. The plunger is received in the barrel. The abutment face of the stopper faces the injection end of the barrel. The elastic jacket includes a through-hole extending along an axis. The elastic jacket is mounted around the plunger. The abutment face of the stopper is exposed outside of the through-hole of the elastic jacket.

Preferably, the stopper includes an outer periphery having an annular groove in which the elastic jacket is engaged.

In a form shown, the abutment face of the stopper includes a maximum width perpendicular to the axis. The maximum width of the abutment face is equal to an inner diameter of the barrel. The abutment face of the stopper includes a contact face at a bottom thereof. The annular groove of the stopper includes a plurality of sections. One of the plurality of sections adjacent to the contact face has an outer diameter smaller than that of the remaining of the plurality of sections.

In another form shown, the maximum width of the abutment face of the stopper in the radial direction is smaller than an inner diameter of the barrel. The annular groove of the stopper includes a plurality of sections. One of the plurality of sections adjacent to the abutment face of the stopper has an outer diameter smaller than that of the remaining of the plurality of sections and is connected to the front end of the stopper. The elastic jacket includes a front end corresponding to the front end of the stopper. An extension extends from the front end of the elastic jacket towards the axis to an extent. The extension abuts the one of the plurality of sections of the annular groove connected to the front end of the stopper.

According to another aspect, the present invention provides a liquid pushing assembly for a syringe with prevention of backflow of blood. The liquid pushing assembly includes a plunger having a stopper on an end thereof. The stopper includes an abutment face. An elastic jacket includes a through-hole extending along an axis. The elastic jacket is mounted around the plunger. The abutment face of the stopper is exposed outside of the through-hole of the elastic jacket. The stopper includes an outer periphery having an annular groove in which the elastic jacket is engaged.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 shows a schematic cross sectional view of a conventional syringe.

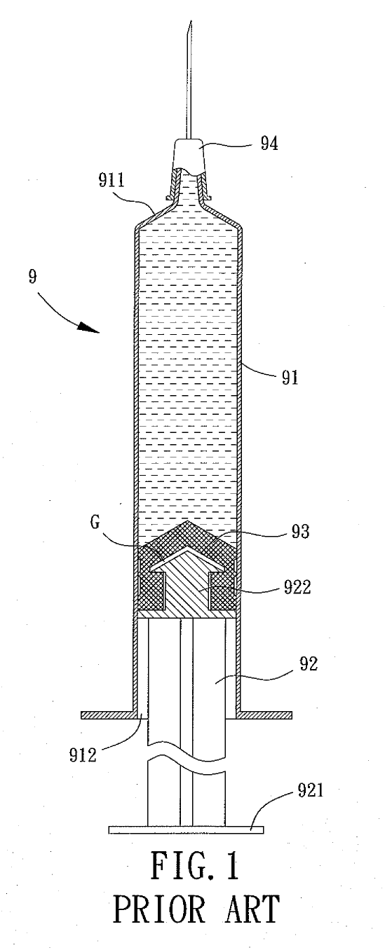

FIG. 2 shows an exploded, perspective view of a syringe with prevention of backflow of blood of a first embodiment according to the present invention, with portions of the syringe broken away.

FIG. 3 shows a cross sectional view of the syringe of FIG. 2.

FIG. 4 is a cross sectional view similar to FIG. 3, wherein a plunger of the syringe is pushed to an extreme position.

FIG. 5 shows an exploded, perspective view of a syringe with prevention of backflow of blood of a second embodiment according to the present invention, with portions of the syringe broken away.

FIG. 6 shows a cross sectional view of the syringe of FIG. 5.

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

Where used in the various figures of the drawings, the same numerals designate the same or similar parts. Furthermore, when the terms “first”, “second”, “front”, “rear”, “inner”, “outer”, “end”, “portion”, “section”, “axial”, “radial”, “annular”, “inward”, “width”, and similar terms are used herein, it should be understood that these terms have reference only to the structure shown in the drawings as it would appear to a person viewing the drawings and are utilized only to facilitate describing the invention.

### DETAILED DESCRIPTION OF THE INVENTION

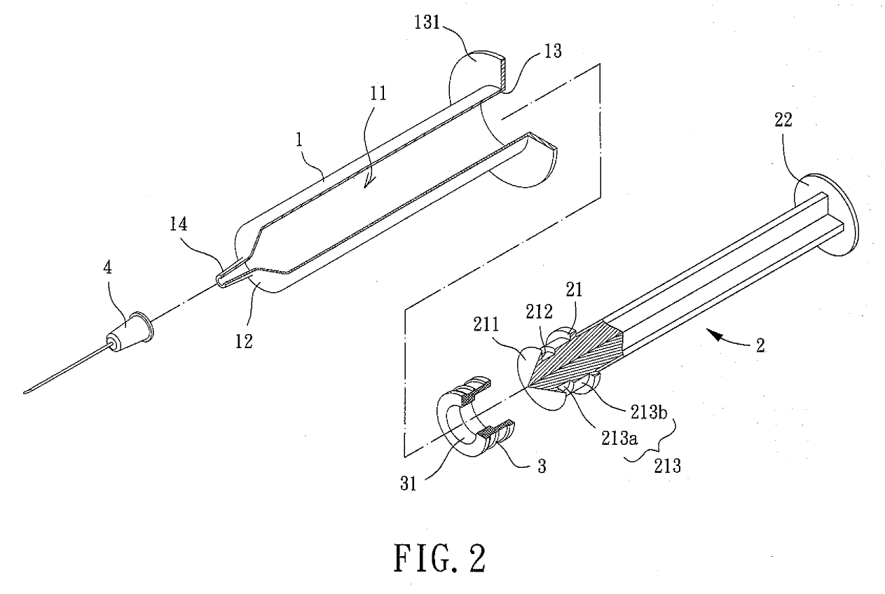

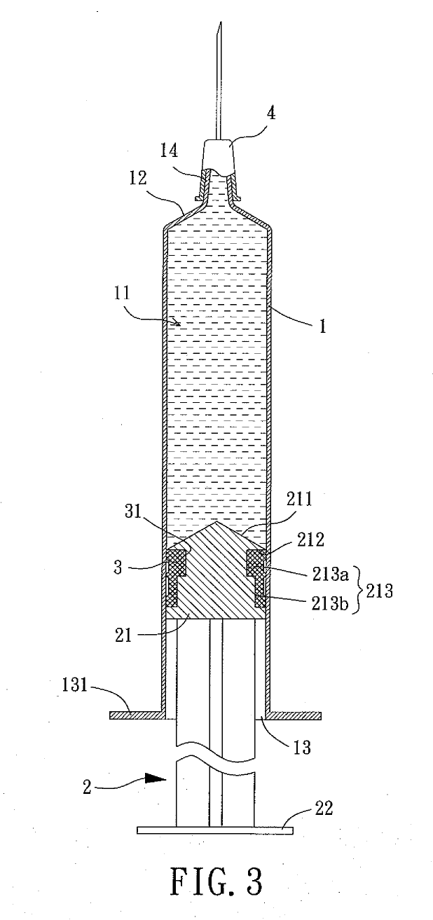

FIGS. 2 and 3 respectively show an exploded perspective view and an assembled cross sectional view of a syringe with prevention of backflow of blood of a first embodiment according to the present invention. In this embodiment, the syringe includes a barrel 1 and a liquid pushing assembly. The liquid pushing assembly includes a plunger 2 and an elastic jacket 3 having an elastic coefficient significantly larger than that of the plunger 2. The elastic jacket 3 is engaged with the plunger 2 that is received in the barrel 1.

The barrel 1 is hollow and includes a compartment 11 for receiving an injection. The barrel 1 includes a tapering end that forms an injection end 12. The barrel 1 further includes an open end 13 spaced from injection end 12 along an axis. In this embodiment, the inner diameter of the injection end 12 tapers away from the open end 13 like a funnel. An engagement section 14 is extended from a portion of the injection end 12 having the smallest inner diameter. A needle cannula 4 is mounted to the engagement section 14. A flange 131 is provided on an outer periphery of the open end 13. A user can hook his or her fingers on the flange 131 while operating the syringe with prevention of backflow of blood.

The plunger 2 includes a stopper 21 on a front end thereof. In this embodiment, the stopper 21 has a fixed outline and is made of a material having a very small elastic coefficient, such as plastic, glass, or the like. The stopper 21 can be integrally formed with the plunger 2 or coupled to the front end of the plunger 2.

The stopper 21 includes an abutment face 211. The plunger 2 is received in the compartment 11 of the barrel 1, with the abutment face 211 of the stopper 21 facing the injection end 12 of the barrel 1. The abutment face 211 of the stopper 21 can abut with an inner periphery of the injection end 12 of the barrel 1. In this embodiment, the injection end 12 of the barrel 1 is in the form of a funnel, and the abutment face 211 of the stopper 21 is in the form of a conic face. The abutment face 211 of the stopper 21 has a maximum width perpendicular to the axis. The maximum width of the abutment face 211 is equal to an inner diameter of the barrel 1. The abutment face 211 of the stopper 21 includes a contact face 212 at a bottom thereof. Preferably, the contact face 212 extends perpendicularly to the axis.

An annular groove 213 is defined in an outer periphery of the stopper 21 for engagement with the elastic jacket 3. The annular groove 213 includes a plurality of sections with differing diameters along the axis. In this embodiment, the annular groove 213 includes a first section 213a and a second section 213b. The first section 213a is adjacent to the contact face 212 and has an outer diameter smaller than that of the second section 213b.

A rear end of the plunger 2 extends outside of the open end 13 of the barrel 1 and includes a thumb rest 22 allowing the user to push the plunger 2 for moving the stopper 21 in the compartment 11 of the barrel 1. In this embodiment, the plunger 2 includes cruciform cross sections to provide structural strength for resisting buckling while pushing the plunger 2. To provide convenient operation, the thumb rest 22 can be a disk coupled to the rear end of the plunger 2. The disk can have any desired shape.

The elastic jacket 3 is made of a material (such as rubber) having an elastic coefficient significantly larger than that of the plunger 2. The elastic jacket 3 includes a through-hole 31 extending along the axis. The elastic jacket 3 is mounted around the stopper 21 of the plunger 2. The abutment face 211 of the stopper 21 is exposed outside of the through-hole 31 of the elastic jacket 3.

The outer diameter of the elastic jacket 3 is equal to or slightly larger than the inner diameter of the barrel 1. The inner diameter of the elastic jacket 3 can be varied to correspond to the sections of the annular groove 213 of the stopper 21. The elastic jacket 3 is mounted in the annular groove 213 of the stopper 21. Some gaps may exist between the engagement area between the elastic jacket 3 and the stopper 21. Alternatively, the elastic jacket 3 can be directly formed in the annular groove 213 of the stopper 21 through injection molding, providing tight engagement between the elastic jacket 3 and the stopper 21.

With reference to FIG. 3, when the front end of the plunger 2 is inserted into the compartment 11 of the barrel 1, the elastic jacket 3 around the stopper 21 can be slightly compressed in a radial direction, such that the stopper 21 abuts with the inner periphery of the barrel 1. The stopper 21 pushes the injection received in the compartment 11 towards the injection end 12 of the barrel 1 to move the injection out of the needle cannula 4 while pushing the plunger 2 in the barrel 1.

Furthermore, since the outer diameter of the first section 213a of the annular groove 213 is smaller, a corresponding portion of the elastic jacket 3 received in the first section 213a has a larger thickness in the radial direction to increase the contact area between the elastic jacket 3 and the contact face 212 of the stopper 21. Thus, the elastic jacket 3 will not disengage from the annular groove 213 of the stopper 21 by the frictional force between the elastic jacket 3 and the inner periphery of the barrel 1 during movement of the plunger 2.

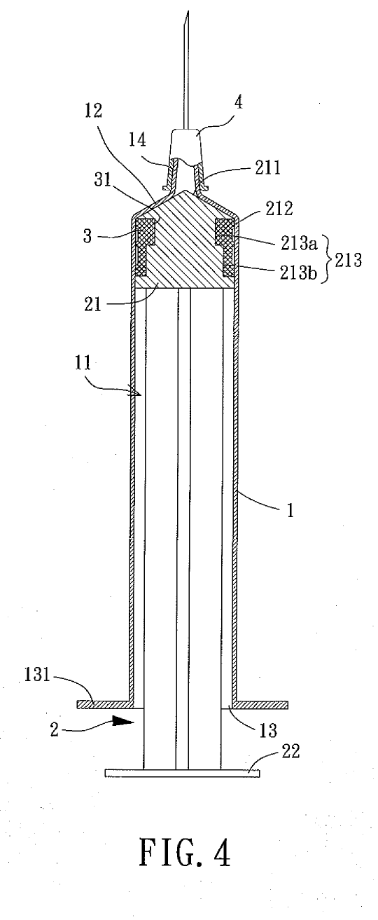

With reference to FIG. 4, when the plunger 2 is moved to its extreme position, the abutment face 211 of the stopper 21 outside of the through-hole 31 of the elastic jacket 3 abuts with the inner periphery of the injection end 12 of the barrel 1. Since the stopper 21 has a very small elastic coefficient and a fixed outline, the abutment face 211 of the stopper 21 abutting with the inner periphery of the injection end 12 of the barrel 1 will not deform by pressure. Thus, no negative pressure will be created at the engagement section 14. By such an arrangement, no suction force is generated in the needle cannula 4 when the user stops pushing the plunger 2, effectively preventing backflow of blood.

Furthermore, since it is the abutment face 211 of the stopper 21 exposed outside of the through-hole 31 of the elastic jacket 3 that abuts with the inner periphery of the injection end 12 of the barrel 1, negative pressure will not be created at the engagement section 14 while pushing the plunger 2, even though gaps exist between the elastic jacket 3 and the stopper 21 due to mounting the elastic jacket 3 in the annular groove 213 of the stopper 21 rather than injection molding, effectively preventing backflow of blood.

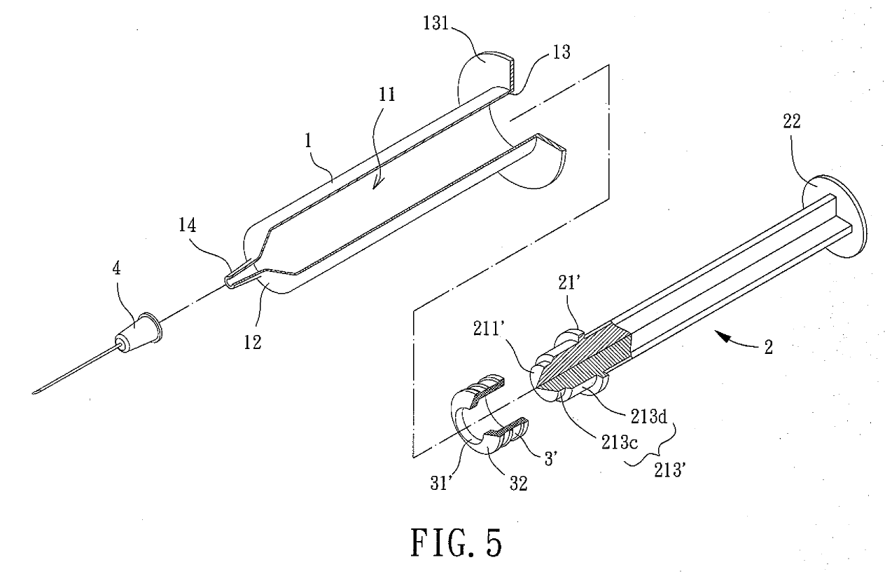

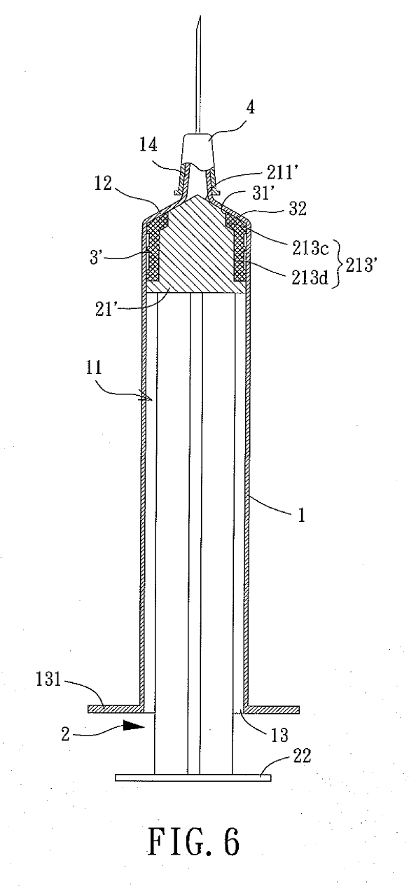

FIGS. 5 and 6 respectively show an exploded perspective view and an assembled cross sectional view of a syringe with prevention of backflow of blood of a second embodiment according to the present invention. The second embodiment is substantially the same as the first embodiment except for the differences in the stopper 21′ and the elastic jacket 3′. The features of the second embodiment identical to those of the first embodiment will not be described to avoid redundancy.

The front end of the stopper 21′ includes an abutment face 211′ that is conical to correspond to the shape of the injection end 12 of the barrel 1. The maximum width of the abutment face 211′ perpendicular to the axis is smaller than the inner diameter of the injection barrel 1. An annular groove 213′ is defined in the outer periphery of the stopper 21′ for engagement with the elastic jacket 3′. The annular groove 213′ includes a plurality of sections with differing diameters along the axis. In this embodiment, the annular groove 213′ includes a third section 213c and a fourth section 213d. The third section 213c is adjacent to the abutment face 211′ and has an outer diameter smaller than the fourth section 213d. Furthermore, the third section 213c is connected to the front end of the stopper 21′.

The elastic jacket 3′ includes an extension 32 extending from an end face of a front end of the elastic jacket 3′ corresponding to the front end of the stopper 21′ towards the axis to an extent. The extension 32 abuts with the third section 213c and maintains the abutment face 211′ of the stopper 21′ to be outside of the through-hole 31′ of the elastic jacket 3′.

With reference to FIG. 6, when the plunger 2 is pushed, the stopper 21′ abuts with the inner periphery of the barrel 1 through the elastic jacket 3′. When the plunger 2 reaches its extreme position, the extension 32 of the elastic jacket 3′ and the abutment face 211′ of the stopper 21′ abut with the inner periphery of the injection end 12 of the barrel 1. In this embodiment, since the stopper 21′ at the engagement section 14 of the barrel 1 has a very small elastic coefficient, effective prevention backflow of blood can also be achieved.

In view of the foregoing, by exposing the abutment face 211, 211′ of the stopper 21, 21′ outside of the through-hole 31, 31′ of the elastic jacket 3, 3′ so that the stopper 21, 21′ having a very small elastic coefficient can abut with the inner periphery of the injection end 12 of the barrel 1, the syringe with prevention of backflow of blood according to the present invention can prevent backflow of blood when the user stops pushing the plunger 2, avoiding blockage of the blood vessel by the blood clot during an intravenous drip. The injection safety is, thus, enhanced.

Furthermore, the abutment face 211, 211′ of the stopper 21, 21′ outside of the through-hole 31, 31′ of the elastic jacket 3, 3′ (rather than the elastic jacket 3, 3′) can directly abut with the inner periphery of the injection end 12 of the barrel 1, the syringe with prevention of backflow of blood according to the present invention can prevent backflow of blood even though axial gaps exist between the stopper 21, 21′ of the plunger 2 and the elastic jacket 3, 3′.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A syringe with prevention of backflow of blood comprising:
a hollow barrel including an injection end; and
a liquid pushing assembly including a plunger and an elastic jacket, with the plunger including a stopper on a front end thereof, with the stopper including an abutment face, with the plunger received in the barrel, with the abutment face of the stopper facing the injection end of the barrel, with the elastic jacket including a through-hole extending along an axis, with the elastic jacket mounted around the plunger, with the abutment face of the stopper exposed outside of the through-hole of the elastic jacket.

2. The syringe with prevention of backflow of blood as claimed in claim 1, with the stopper including an outer periphery having an annular groove, with the elastic jacket engaged in the annular groove.

3. The syringe with prevention of backflow of blood as claimed in claim 2, with the abutment face of the stopper including a maximum width perpendicular to the axis, with the maximum width of the abutment face equal to an inner diameter of the barrel, with the abutment face of the stopper including a contact face at a bottom thereof.

4. The syringe with prevention of backflow of blood as claimed in claim 3, with the annular groove of the stopper including a plurality of sections, with one of the plurality of sections adjacent to the contact face having an outer diameter smaller than that of the remaining of the plurality of sections.

5. The syringe with prevention of backflow of blood as claimed in claim 2, with the maximum width of the abutment face of the stopper in the radial direction smaller than an inner diameter of the barrel.

6. The syringe with prevention of backflow of blood as claimed in claim 5, with the annular groove of the stopper including a plurality of sections, with one of the plurality of sections adjacent to the abutment face of the stopper having an outer diameter smaller than that of the remaining of the plurality of sections and connected to the front end of the stopper.

7. The syringe with prevention of backflow of blood as claimed in claim 6, with the elastic jacket including a front end corresponding to the front end of the stopper, with an extension extending from the front end of the elastic jacket towards the axis to an extent, with the extension abutting the one of the plurality of sections of the annular groove connected to the front end of the stopper.

8. A liquid pushing assembly for a syringe with prevention of backflow of blood as comprising:
a plunger including a stopper on an end thereof, with the stopper including an abutment face; and
an elastic jacket including a through-hole extending along an axis, with the elastic jacket mounted around the plunger, with the abutment face of the stopper exposed outside of the through-hole of the elastic jacket.

9. The liquid pushing assembly for a syringe with prevention of backflow of blood as claimed in claim 8, with the stopper including an outer periphery having an annular groove, with the elastic jacket engaged in the annular groove.
